(19) United States
(12) Patent Application Publication
(10) Pub. No.: US 2004/0138607 A1
(43) Pub. Date: Jul. 15, 2004

(54) Cartridge-based medical fluid processing system

(76) Inventors: Jeffrey H. Burbank, Boxford, MA (US); James M. Brugger, Newburyport, MA (US); Dennis M. Treu, Bedford, NH (US); William K. Weigel, York, ME (US)

Correspondence Address: PROSKAUER ROSE LLP, PATENT DEPARTMENT, 1585 BROADWAY, NEW YORK, NY, 10036-8299, US

(21) Appl. No.: 10/680,889

(22) Filed: Oct. 8, 2003

Related U.S. Application Data
(60) Provisional application No. 60/417,377, filed on Oct. 8, 2002.

(51) Int. Cl. A61M 37/00; C02F 9/00; B01D 1/00

(52) U.S. Cl. 604/6.13; 604/4.01; 210/257.1; 210/646

(57) ABSTRACT

A blood processing system ensures proper temperature of blood returned to a patient by means of a fluid circuit with a warming portion that is engaged with the entirety of the fluid circuit in a single operational step. In an embodiment, the single-step set up is achieved by providing a cartridge-mounted fluid circuit with a warming portion integrated into it. A blood processing machine engages actuators, sensors, etc. of the blood processing and simultaneously a heating portion to supply heat to the blood circuit in a manner that ensures a proper return temperature of blood.

### RELATED APPLICATIONS

The present application claims priority to U.S. Provisional Application Ser. No. 60/417,377 filed Oct. 8, 2002 and entitled: “Cartridge-based medical fluid processing system.”

### BACKGROUND

As part of treating certain medical conditions, it is often beneficial to infuse fluids into a patient. Systems that infuse may involve extraction as well, for example, hemodialysis, hemofiltration, apheresis, and others. The latter category, extracorporeal blood treatments, remove blood from a patient, to process or treat it in some manner, and subsequently return the processed blood to the patient's body. In dialysis treatment, for example, waste products are removed from the blood to compensate for kidney failure. Blood processing may also be performed in healthy patients. For example, when a person donates platelets, the donor's blood is removed from the donor's body, platelets are extracted, and the blood is subsequently returned to the donor's body.

While the majority of bodily fluid processing systems are blood processing systems, fluid processing systems may also be used to process other bodily fluids (e.g. lymphatic fluid, spinal fluid, etc.). For the sake of convenience, the embodiments described herein will assume that the fluid being processed is blood. As will be apparent to persons skilled in the relevant arts, however, these teachings may be applied with equal force to the processing of any other bodily fluid.

Fluid processing machines are generally complex, and great care must be taken to prevent contamination of the blood that is ultimately returned to the patient's body. Failure to take appropriate steps to prevent such contamination can interfere with the treatment that is being performed. In some cases, it can even introduce a harmful agent to the patient's body.

One popular technique for avoiding contamination of the patient's blood during processing is to partition the blood processing system into a base unit and a removable fluid circuit including bodily fluid and blood portions, for example, or simply an infusate circuit. In such systems, the entire fluid circuit may be disposable to prevent the possibility of cross-contamination between one patient and another. Fluid circuits are sometimes held in some sort of holder or cartridge to position various portions of the circuit relative to sensors, actuators, and pumps on the base unit.

A common example of an actuator that is used in such base systems is a shutoff valve that selectively pinches tubing through which fluid flows, thereby selectively impeding the fluid flow or allowing it to flow unimpeded. Other examples of actuators include pressure regulators, pressure sensor actuators, and volume chambers. A common example of a pump is a peristaltic pumps that moves fluid through tubing in the cartridge using a rotating set of rollers that do not break the hermetic seal of the sterile disposable fluid circuit. Examples of common sensors include temperature sensors, and air sensors.

In some applications, a significant volume of fluid is removed from the blood during processing, and this fluid must be replaced to prevent the patient from dehydrating. This is typically accomplished by feeding a supply of replacement fluid into the blood processing system, and controlling the feed volume so that the volume of fluid returned to the patient's body is approximately the same as the volume that was removed.

FIG. 1 is a figurative representation of a blood processing cartridge 110. The cartridge includes a filter 130, an input tube 132 that supplies the blood to the filter 130, an output tube 134 that provides a return path for the filtered blood, and a waste output tube 132 through which undesired waste products are removed. The cartridge also includes a balance chamber 120 which acts as a pump for the replacement fluid. The balance chamber 120 is filled via fluid input line 164, and emptied via the fluid output tube 122. Ultimately, the blood from the blood out tube 134 and the replacement fluid from the replacement fluid out tube 122 are returned to the patient's body using known fluid balancing techniques.

In certain cases, particularly when a relatively large volume of replacement fluid must be added, the replacement fluid must be warmed before it is returned to the patient's body. The conventional approach to warming the replacement fluid is by using a warmer (e.g., an electric heater) 150. FIG. 1 depicts an example of such a heat exchanger. Replacement fluid enters the warmer 150 through tubing 162. The fluid then flows into a serpentine tubing portion or heat exchanger 160, which provides a relatively large surface area through which heat transfer can be effected. The serpentine tubing 160 is mounted on the warmer's chassis for ease of handling. The warmed fluid eventually flows out through fluid line 164 for subsequent processing in cartridge 110.

Notably, in the prior art configuration, the body of the cartridge 110 and the body of the warmer 150 are discrete components, and each of those components may require a separate installation procedure: one operation to hook up the cartridge, and a separate operation one to hook up the warmer. The need to perform two separate hook-up and engagement operations for the warmer and for the cartridge is labor-intensive and creates opportunities for the introduction of faults in the mechanical configuration.

The prior art arrangement, in which the cartridge and the warmer are separate, also suffers from additional disadvantages. In many cases, heat that is generated during normal operation of the blood processing system must be removed to keep the system working properly. This is typically accomplished using fans, which transfer the heat from the system to the surrounding room. Thus, we have a situation in which energy is expended to remove heat from the blood processing machine, while energy is simultaneously being expended to add heat to the replacement fluid. This wastes energy, and adds heat to the room in which the blood processing machine is being used, which may make the room uncomfortable.

The inventors have recognized a need to avoid the aforementioned disadvantages.

### SUMMARY OF THE INVENTION

One aspect of the present invention relates to implementing both the fluid processing and heat exchange functions in a single cartridge. This can simplify the set up of the equipment because fewer set up and installation operations are necessary. In certain embodiments, it may even be possible to install both the heat exchange components and the fluid processing components in a single operation (e.g., by closing the door of a cartridge bay). It can also simplify post-processing labor and minimize the amount of things that must be disposed subsequent to use.

A second aspect of the invention relates to using the heat generated by the blood processing machinery, that would otherwise be wasted, to warm the replacement fluid that is returned to the patient's body. This aspect of the invention can provide two advantages. Reduction in energy consumption, and a reduction in the amount of waste heat generated by the machine.

### BRIEF DESCRIPTION OF THE DRAWINGS

FIG. 1 is a schematic representation of a prior art blood processing cartridge and a separate prior art heat exchanger.

FIG. 2 is a schematic representation of a blood processing cartridge in accordance with an embodiment of the present invention.

FIG. 3 is a schematic representation of a heat exchanging surface of a blood processing base unit, that is designed to meet with the cartridge of FIG. 2.

FIG. 4 is a side view of the heat exchanging surface of FIG. 3 and a side view of the cartridge of FIG. 2, before those two components are fastened together.

FIG. 5 is a side view of the heat exchanging surface of FIG. 3 and a side view of the cartridge of FIG. 2, after those two components are fastened together.

FIG. 6 is a block diagram of a heat management system in a blood processing base unit that includes the heat exchanging surface of FIG. 2.

FIG. 7 is a block diagram of an alternative heat management system in a blood processing base unit that includes the heat exchanging surface of FIG. 2.

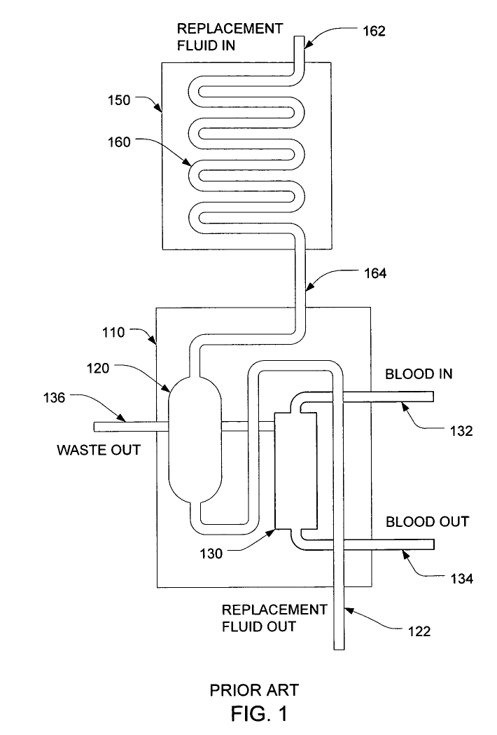

### DETAILED DESCRIPTION OF THE PREFERRED EMBODIMENTS

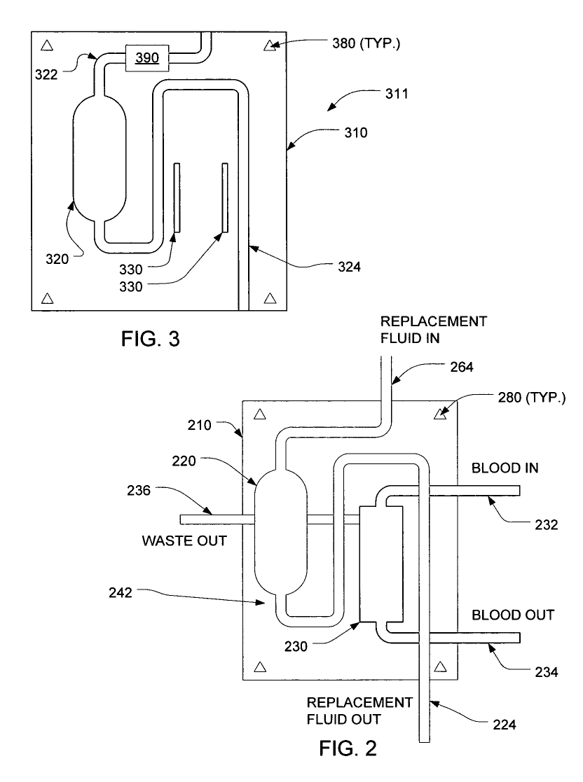

FIG. 2 is a schematic representation of a blood processing cartridge 210 for use with an infusion or treatment machine. These may be, for example a blood processing system with features such as described in the following patent applications which are hereby incorporated by reference as if fully set forth in their entirety herein:

U.S. patent Ser. No. 09/513,902 filed Feb. 25, 2000 entitled: Layered Fluid Circuit Assemblies and Methods For Making Them; U.S. patent Ser. No. 09/513,773 filed Feb. 25, 2000 entitled: Fluid Processing Systems and Methods Using Extracorporeal Fluid Flow Panels Oriented Within A Cartridge; U.S. patent Ser. No. 09/513,911 filed Feb. 25, 2000 entitled: Synchronized Volumetric Fluid Balancing Systems and Methods; U.S. patent Ser. No. 09/513,564 filed Feb. 25, 2000 entitled: Systems and Methods for Performing Frequent Hemofiltration, Arterial Pressure/Air Sensing; and U.S. patent Ser. No. 09/451,238 filed Nov. 29, 1999 entitled: Systems and Methods for Performing Frequent Hemofiltration. The above references describe extracorporeal blood treatment devices, but the embodiments disclosed in the present application are applicable to drug and fluid infusion systems, transfusion devices, heart-lung machines, etc. as will be apparent from the following disclosure. The cartridge includes a cartridge body upon which a fluid circuit 244 is mounted. Blood from the patient arrives via the blood input tube 232 and is processed by the filter 230. Any suitable medical grade tubing may be used for this application (and for all other blood transport applications described herein). A filter 230 may be present to remove fluid from the blood. The removed waste flows out of the filter 230 via the waste out line 236. Typically, a significant quantity of fluid will flow out of the waste out tube 236 in addition to the waste components themselves. All remaining portions of the blood that have not been eliminated via the waste out line 236 will flow out of the filter 230 via the blood out line 234, and ultimately returned to the patient.

In order to compensate for loss of fluid volume, which may be a desired effect of the filtering process, a quantity of replacement fluid must be returned to the patient in addition to the blood that is being returned via the blood out line 234. Adding replacement fluid to the return blood may be implemented in any conventional manner, such as using the balance chamber 220 illustrated schematically in FIG. 2. Replacement fluid arrives at the balance chamber 220 via the input tube 264. Any conventional source of replacement fluid may be used to supply the input fluid line 264. The output of the balance chamber 220 is routed via the fluid out line 224. The replacement fluid that travels through the return fluid line 224 is ultimately combined with the blood from the return blood line 234 and returned to the patient's body.

The illustrated cartridge 210 also includes fasteners 280 to hold the cartridge 210 onto the base unit 311. These fasteners are configured to mate with corresponding fasteners on the base unit 311. In alternative embodiments, fasteners may be omitted from the cartridge 210, provided that the base unit 311 is appropriately designed (as shown, for example, in FIG. 9).

Although only one example configuration for a cartridge is shown in FIG. 2, numerous alternatives configurations could also be used as will be recognized by persons skilled in the art, depending on the functions to be performed by the cartridge 210. For example, fluid balancing may be performed by electronic flow measurement and direct control of the replacement fluid stream, by mass-balancing, either mechanical or electronically controlled, as well as other methods.

FIG. 3 is a schematic representation of a heat exchanging surface of a blood processing base unit 311 that is adapted to accept the cartridge of FIG. 2. This heat exchanging surface is a part of the base unit 311 of the blood processing machine. It is designed so that when the cartridge 210 makes thermal contact with the heat exchange surface 310, a heat transfer path is established between the heat exchange surface 310 and the fluid contained in the balance chamber 220 and/or the fluid lines 222, 224. To facilitate efficient heat transfer, the heat exchange surface 310 is preferably made out of a solid heat conductive material (e.g., a metal such as aluminum or copper).

The heat exchange surface 310 is designed to make effect thermal contact with the fluid circuit 244 of the cartridge of FIG. 2. In the illustrated configuration, it also includes a pair of filter-guides 330 that guide the filter 220 (on the cartridge) into position.

The heat exchange surface 310 also includes a channel 320 that is shaped to accept the balance chamber 220 of the cartridge. Optionally, the heat exchange surface 310 may also include smaller channels 322, 324 that are configured to align with the fluid lines 222, 224 when the cartridge 210 is fastened to the heat exchange surface 310. In the illustrated embodiment, the channel 320 is concave, with a concavity designed to match the convex outer surface of the balance chamber 220 of the cartridge.

The heat exchange surface 310 also includes a set of fasteners 380 that are configured to engage with fasteners 280 of the cartridge. Alternatively the fluid circuit portion of the cartridge may be self-locating by engaging various fluid circuit portions with corresponding engagement elements such as pumps, actuators, supports, clamps etc.

When the cartridge 210 is fastened to the heat exchange surface 310, the heat-conducting material of the heat exchange surface preferably presses against the various components of the cartridge 210 including the fluid circuit 242. Thus, heat can be transferred into the replacement fluid contained in the cartridge 320 by heating the heat exchange surface 310.

In one preferred embodiment, the wall of the balance chamber 220 that comes in contact with the heat exchange surface 310 when the cartridge 210 is fastened to the heat exchange surface 310 is made of a flexible non-porous material that also conducts heat. When a balance chamber 220 with a flexible outer wall is used, it will conform to the shape of the heat exchange surface 310 when the cartridge 210 is fastened to the heat exchange surface 310 and fluid pressure inflates it. This conformation helps to provide a large contact area and is effective for thermal contact between the heat exchange surface 310 and the wall of the balance chamber 220. Optionally, the concave portion of the heat exchange surface and/or the convex surface of the balance chamber 220 may be coated with a heat-conducting material (e.g., thermal grease) in order to improve the heat transfer characteristics of the interface. For more rigid lines or vessels, the heat exchange surface may be provided with a flexible conforming surface such as by using thin resilient metal.

In addition to providing a thermal path to heat the fluid in the fluid circuit 244, the base unit 311 drives the cartridge 210 by operating drivers 390 (i.e., actuators and/or pumps). The drivers 390 are configured so that when the cartridge 210 is engaged with the heat exchange surface 310, the drivers can drive the fluid circuit 242 in a manner that causes the fluid circuit 242 and related components such as base unit 311 to perform an intended operation. The particular drivers used in any given case will, of course, depend on the configuration and the base unit 311 and the overall function being performed by the treatment machine. In addition, the cartridge 210 is configured in a corresponding manner so it can be driven by the drivers 390. Numerous examples of fluid circuit-driving arrangements can be readily envisioned, such as pumping fluids and selectively clamping lines. For example, the driver 390 may be a peristaltic pump that engages with the fluid line 222 of the fluid circuit 242 in order to pump replacement fluid into the balance chamber 220. Because of this wide variation, the elements that drive the fluid circuit 244 are schematically represented by the box labeled driver 390.

Optionally, the base unit 311 can base its control of the cartridge-driving operations by using feedback from sensors (not shown) that sense certain conditions in the cartridge 210. Examples of such sensors include temperature sensors, pressure sensors, sensors that detect air in the fluid lines., etc. The output of these sensors are inputted by the base unit 311, and the base unit modifies its driving operations in response.

The fasteners 380 are configured to mate with corresponding fasteners on the cartridge base unit 311 so as to hold the cartridge 210 onto the base unit 311. Any of a wide variety of fastening systems may be used for this purpose, including but not limited to snaps, Velcro™, wingnuts, clamps, screws, closing a door onto the cartridge (where the door latches in the shut position), etc. Of course, while only one example configuration for the heat exchange surface is shown in FIG. 3, numerous alternatives configurations could also be used, as long as they interface correctly with the cartridge that is being used.

Preferably, when the cartridge 210 is fastened onto the base unit 311, the fastening operation performs two functions: it engages the thermal transferring surfaces of the cartridge 210 and the heat exchange surface 310 of the base unit, and also engages the drivers 390 with the corresponding components of the cartridge 210. This simplifies the installation procedure as compared to systems that require one fastening operation for heat exchange, and a second fastening operation for the interface that drives the cartridge.

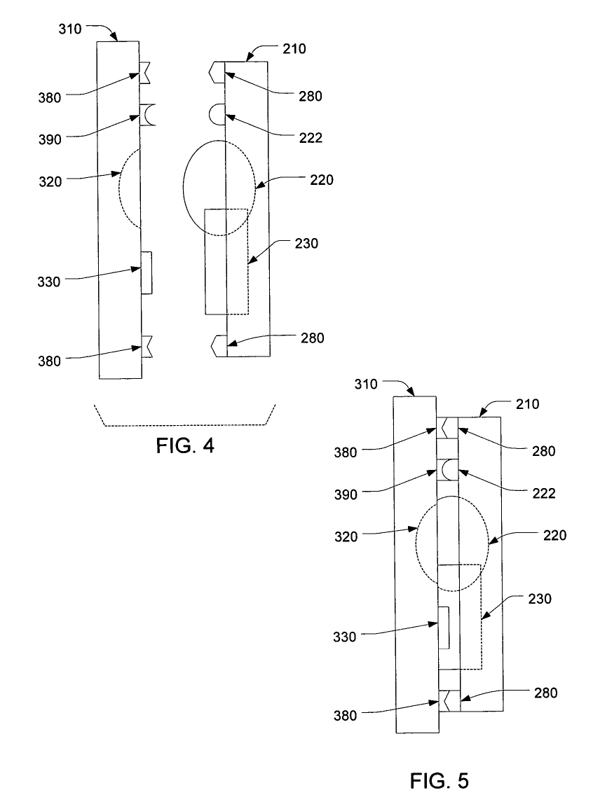

FIG. 4 is a side view that shows how the heat exchange surface 310 may mate with the cartridge 220 and fluid circuit 242, just before those components have mated. Note that when the same reference number is used herein in two different figures, that reference number represents the same item in both figures. FIG. 4 schematically shows how the concave well 320 in the heat exchange surface 310 is designed to fit the convex surface of the balance chamber 220. It also shows schematically how the fasteners 280 are designed to mate up with the fasteners 380, and how the guides 330 are aligned to guide the filter 230 into place. The driver 390 aligns with a correspondingly located fluid line 222.

FIG. 5 is a schematic illustration of what happens when the heat exchange surface (the left side of FIG. 4) and the cartridge (the right side of FIG. 4) are fastened together. The mating of the fasteners 280, 380 is schematically illustrated by the mating of the male and female triangle-shaped objects, and the mating of the driver with the fluid line is schematically represented by the mating male and female semicircular objects 222, 390. The cartridge may have a surface that is molded to match precisely the heat exchange surface or an opening may be provided in the cartridge to allow effective thermal contact between the balance chamber and heat exchange surface 310.

When the heat exchange surface 310 is fastened to the cartridge 210, thermal conductivity is established by conforming the balance chamber 220 to the heat exchange surface 310 and the cartridge 210. This conformation is schematically illustrated by the fact that the radius of the shape channel 320 is shown as permitting a good match to the balance chamber 220. Because of this, when the cartridge 210 is fastened to the heat exchange surface 310, the balance chamber 220 will be pressed against the heat exchange surface so that heat is transferred from the heat exchange surface 310 to the contents of the balance chamber 220 more effectively.

### ******** INSERT FROM TAPE **********

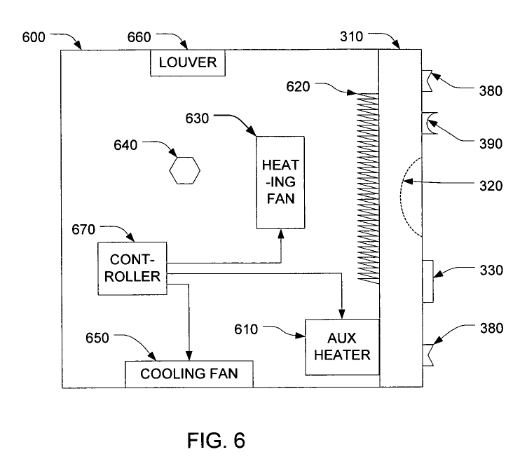

FIG. 6 shows the heat exchange surface 310 (previously discussed in connection with FIGS. 4 and 5) attached to the side of the blood processing base unit 600. A heat sink 620 is thermally connected to the heat exchange surface 310. In contrast to conventional electronic applications, where heat sinks are used to extract heat from a component, here the heat sink 620 is being used to add heat to the heat exchange surface 310.

The hexagon 640 represents components of the fluid processing machine that generate heat. The heating fan 630 circulates the air within the cabinet of the fluid processing machine 600. The circulating air cools the heat generating component 640 and raise the temperature of the air inside the cabinet 600. The heat sink 620 absorbs heat from the circulating air so that it can be transferred out into the fluid by conduction through the heat exchange surface 310. An auxiliary heater 610 is also provided to heat the fluid in cases where the heat generating components 640 do not generate sufficient heat to raise the temperature of the fluid.

In the illustrated embodiment, a controller 670 is used to turn the heating fan 630 and/or the auxiliary heater 610 on and off to heat up the heat exchange surface 310 as required. Optionally, the controller 670 may be provided with temperature information that describes one or more of the following temperature parameters: the temperature of the replacement fluid arriving from its original source (e.g., a hang bag, not shown); the temperature of the replacement fluid near the point of delivery to the patient; the temperature of the fluid being processed in the cartridge, the internal temperature of the cabinet 600 of the fluid processing machine, and the temperature of the room where the fluid processing operation is taking place. The air temperatures may be sensed, for example, using an appropriate integrated circuit such as the National Semiconductor LM335 temperature sensor. The fluid temperatures may be sensed using techniques that are well known in the medical fluid processing field, such as by a contact thermistor or other electronic temperature probe that may be wetted or surface contact type with insulation to ensure accurate temperature readings. Based on the temperature information, the controller 670 determines when to actuate the heating fan 630 or the auxiliary heater 610 to achieve the desired medically indicated condition, such as a proper infusion temperature for replacement fluid.

Note that although the above discussion relates to warming of replacement fluid, it applies also to infusate of any kind. In addition, the sensed temperature upon which operation depends may be the temperature of blood at the point of the patient access. The latter may be appropriate where blood may be cooled by its course through treatment equipment. Thus, the control may be such as to inject replacement fluid into returning blood at a temperature that brings the blood/replacement fluid mixture to the desired temperature.

Optionally, a cooling fan 650 may also be provided to cool the internal components of the fluid processing machine 600 to prevent the system from overheating. For example, an exhaust fan 650 may be configured to pull air in through a louver 660, so that it passes over the heat-generating components 640. Optionally, operation of the cooling fan 650 may also be controlled by the controller 670. For example, the controller 670 may be programmed to turn the cooling fan on whenever the internal cabinet is too hot (e.g., when it exceeds a preset value such as 150° F.). In alternative embodiments, the cooling fan may be thermostat-controlled.

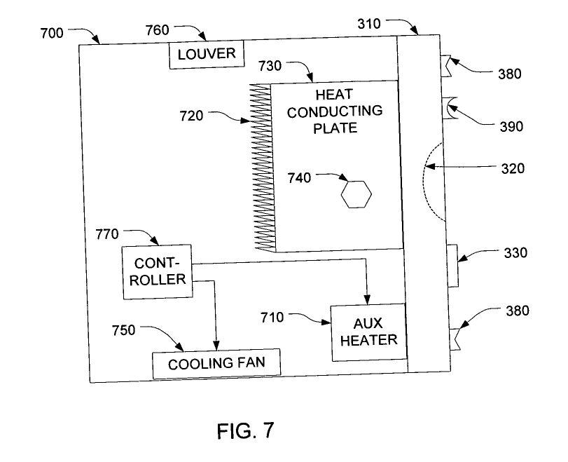

FIG. 7 shows the heat exchange surface 310 (previously discussed in connection with FIGS. 4 and 5) attached to the side of an alternative blood processing base unit 700. The hexagon 740 represents components of the fluid processing machine that generate heat. It is mounted on a heat conducting plate 730, which conducts heat from this heat-generating component 740 to the heat exchange surface 310 (for the purpose of heating the fluid). An auxiliary heater 710 is also provided to heat the fluid in cases where the heat generating components 740 do not generate sufficient heat to raise the temperature of the fluid. A controller 770 is used to turn on and off the auxiliary heater 710 to heat up the heat exchange surface 310 as required.

A cooling fan 750 is also provided to cool the internal components of the fluid processing machine 700 to prevent the system from overheating. For example, an exhaust fan 750 may be configured to pull air in through a louver 760, so that it passes over the heat generating components 740. Here, unlike the FIG. 6 embodiments, the heat sink 720 is used to remove heat from the heat-generating component 740, to slow down the transfer of heat into the heat exchange surface 310.

Optionally, the controller 770 may be provided with temperature information similar to the controller described in connection with the FIG. 6 embodiment. Based on the temperature information, the controller 770 determine when to actuate the auxiliary heater 710 and the cooling fan 750.

Note that in the above embodiments, air is used as an intermediary for exchange of heat between heat-generating components and a heat transfer surface forming part of a treatment apparatus. In alternative embodiments, it is also possible to force warm air over the fluid circuit directly rather than relying on conduction through a heat transfer surface. Alternatively, it may be possible to connect heat generating components directly to a heat generating surface that is in contact with a fluid circuit thereby avoiding reliance on convective heat transfer from the heat generating components to the heat transfer surface such as heat conducting plate heat conducting plate 730 or heat exchange surface 310. In still other alternative embodiment, a heat-conducting fluid such as circulating oil may be used in the thermal path between the heat generating component 740 and the heat exchange surface 310.

Some of the embodiments described above advantageously combine the fluid-warmer into the blood processing cartridge. This simplifies the installation procedure, since a single cartridge performs both the blood processing function and the heat exchanging function. The need to perform two separate hook-up operations and two separate disposal operations (i.e., for the heat exchanger and for the cartridge) is therefore eliminated.

Some of the embodiments described above provide improved energy efficiency and thermal management, since heat that is generated during normal operation of the blood processing system is transferred to the blood (which simultaneously heats the blood and cools the processing unit).

We claim:

1. A processing base unit that interfaces with a fluid-processing cartridge for extracorporeal blood processing, the base unit comprising:
a heat exchange surface; and
a cartridge interface configured to interface with the fluid-processing cartridge so that when the fluid-processing cartridge is installed, the cartridge interface (a) actuates the fluid-processing cartridge such that the fluid-processing cartridge processes a fluid and (b) holds the fluid-processing cartridge in a position that keeps the heat exchange surface in thermal contact with the fluid,
wherein the cartridge interface is configured so that (a) portions of the cartridge interface that drive the fluid-processing cartridge and (b) portions of the cartridge interface that keeps the heat exchange surface in thermal contact with the fluid are both engaged in response to a single operator action.

2. The fluid processing base unit of claim 1, wherein the base unit further comprises a fastening system, and the single operator action comprises the step of engaging the fastening system.

3. The fluid processing base unit of claim 2, wherein fastening system comprises a lever, and the operator engages the fastening system by moving the lever.

4. The fluid processing base unit of claim 1, wherein the single operator action comprises the step of engaging a fastening system.

5. The fluid processing base unit of claim 1, wherein when the fluid-processing cartridge is installed, heat transfer from the heat exchange surface to the fluid is regulated.

6. A cartridge for processing a fluid comprising:
a thermal-transfer surface that is maintained in thermal contact with the fluid that is being processed; and
at least one control surface {provide AB in spec} that, when driven in an appropriate manner, causes the cartridge to process the fluid,
wherein the cartridge is configured to fit in or on a compatible fluid processing base unit so that the fluid processing base unit can (a) drive the at least one control surface in the appropriate manner and (b) transfer heat to the thermal-transfer surface, and
wherein the cartridge is configured so that a single operator action engages the at least one control surface and the thermal-transfer surface with corresponding portions of the fluid processing base unit.

7. The cartridge of claim 6, wherein the single operator action comprises the step of engaging a fastening system.

8. The cartridge of claim 6, wherein the thermal-transfer surface is configured so that when the cartridge is installed in or on the fluid processing base unit having a heat exchange surface, the thermal-transfer surface presses against the heat exchange surface and conforms to the shape of the heat exchange surface.

9. The cartridge of claim 8, wherein the thermal-transfer surface is flexible.

10. A fluid processing system comprising:
a base unit;
a cartridge for processing a fluid; and
a fastening system that holds the cartridge in or on the base unit,
wherein the cartridge includes a thermal-transfer surface that is in thermal contact with the fluid that is being processed, and at least one control surface that, when driven, causes the cartridge to process the fluid,
wherein the base unit includes a heat exchange surface and at least one driver configured so that when the cartridge is held in or on the base unit by the fastening system, (a) the heat exchange surface is kept in thermal contact with the thermal-transfer surface, and (b) the at least one driver can drive the at least one control surface in the appropriate manner, and
wherein the base unit and the cartridge are configured so that fastening system can be fastened by a single operator action that engages the at least one driver with the at least one control surface and also brings the heat exchange surface into thermal contact with the thermal-transfer surface.

11. The fluid processing system of claim 10, wherein heat transfer from the heat exchange surface to the fluid is regulated.

12. The fluid processing system in which the of claim 10, wherein heat transfer from the base unit into the cartridge is relied upon to cool the base unit.

13. The fluid processing system of claim 10, wherein the fastening system is integrated into the base unit, and the single operator action comprises the step of engaging the fastening system.

14. The fluid processing system of claim 10, wherein a first part of the fastening system is integrated into the base unit, and a second part of the fastening system is integrated into the cartridge.

15. An apparatus for processing medical fluids prior to their introduction into a patient's body, the apparatus comprising:
a fluid path, the path having a fluid input and a fluid output, wherein the fluid path is configured so that medical fluid can be introduced into the patient's body via the fluid output; {Spec: there may be intermediate structures.}
a component that performs a function other than heat generation, wherein the component generates heat as a by-product of performing the function;
a thermal path having low thermal resistance that transfers at least some of the heat generated by the component to the fluid path;
a first temperature sensor that senses the temperature of the fluid path (at input or return, or indirect at heat sink)
a heater that is thermally connected to the fluid path via a path with low thermal resistance; and
a control system that activates the heater when the temperature sensed by the first temperature sensor is too low. (spec thermostat on/off, T sensor+variable heat out)

16. The apparatus of claim 15, wherein the thermal path comprises a thermally conductive metal that is formed in a thermally conductive shape.

17. The apparatus of claim 15, wherein the thermal path comprises a fan and at least one heat sink.

18. The apparatus of claim 15, wherein the component is located inside a cabinet, and wherein the apparatus further comprises:
a second temperature sensor that senses the temperature at a location inside the cabinet; and
a cooling system configured to cool the component when the temperature inside the cabinet is set beyond a predetermined level.

19. A method for processing a medical fluid prior to the fluid's introduction into a patient's body, the method comprising:
processing the medical fluid using a mechanism that generates heat as a by-product;
providing a thermal path through which the heat generated in the processing step can flow efficiently into the medical fluid;
heating the medical fluid by allowing the heat generated in the processing step to flow into the medical fluid via the thermal path; and
introducing the medical fluid that was heated in the heating step into the patient's body.

20. The method of claim 19, further comprising the steps of:
sensing the temperature of the medical fluid; and
adding heat to the medical fluid using an auxiliary heater when the temperature sensed in the sensing step is too low.

21. The method of claim 19, further comprising the steps of:
(a) sensing the temperature in a cabinet that houses the mechanism; and
(b) cooling the mechanism when the temperature sensed in step (a) is too high.
